(19) United States
(12) Patent Application Publication
(10) Pub. No.: US 2006/0095137 A1
(43) Pub. Date: May 4, 2006

(54) Nanofibrous nonwoven membrane of silk fibroin for guided bone tissue regeneration and manufacturing method thereof

(75) Inventors: Chong-Pyoung Chung, Seoul (KR); Won-Ho Park, Daejeon (KR); Kyoung-Hwa Kim, Goyang-si (KR); Lim Jeong, Daejeon (KR)

Correspondence Address: BACHMAN & LAPOINTE, P.C., 900 CHAPEL STREET, SUITE 1201, NEW HAVEN, CT, 06510, US

(73) Assignee: Seoul National University Industry Foundation

(21) Appl. No.: 11/229,138

(22) Filed: Sep. 16, 2005

(30) Foreign Application Priority Data
Oct. 29, 2004 (KR) 2004-0087254

(51) Int. Cl. A61F 2/28; C12N 5/08

(52) U.S. Cl. 623/23.58; 623/23.76; 435/399; 264/28; 264/465

(57) ABSTRACT

The present invention relates to a membrane for guided bone tissue regeneration and, more particularly, to a membrane for guided bone tissue regeneration having a structure that silk fibroin nanofibers obtained by removing sericin from silk fibers are formed as a nonwoven, and a manufacturing method thereof. A membrane for guided bone tissue regeneration according to the present invention has a predetermined strength, biocompatibility, and biodegradability, and may maintain a sustained drug release system, when drugs are added in the manufacturing process. Additionally, a membrane for guided bone tissue regeneration according to the present invention may be modified corresponding to the condition of usage, because a thickness of the membrane may be adjusted by controlling fineness of nanofibers, compactness of nanofibers, and pore size of a multiporous structure may be adjusted, in a nonwoven manufacturing process. A nanofibrous membrane for guided bone tissue regeneration according to the present invention is manufactured by freezing rapidly, drying a silk fibroin solution obtained by removing sericin from silk fibers, and by electrospinning after dissolving the dried silk fibroin in an electrospinning solvent. The membrane according to the present invention has excellent adhesion and air permeability, and is thereby effective in regeneration of damaged periodontal tissues.

### BACKGROUND OF THE INVENTION

1. Field of the Invention

The present invention relates to a membrane for guided bone tissue regeneration and, more particularly, to a membrane for guided bone tissue regeneration having a structure that nanofibers of silk fibroin obtained by removing sericin from silk fibers are formed as a nonwoven, and a manufacturing method thereof.

2. Description of the Related Art

A method of inducing solidification by filling a damaged region with autografting is available for regeneration of an alveoli damaged by periodontal diseases. As an alternative method, a human bone or animal bone with removed immunogenicity, or commercially available hydroxyapatite is used as an artificial bone replacement material.

Recently, researches are actively carried out for improving curing effect of damaged periodontal tissues by introducing an artificial membrane into the tissues, for improving bone graft and restoring to integral periodontal tissues, and for inducing generation of new alveoli. A membrane used in this technology provides generation of new alveoli and periodontal ligament tissues by isolating a damaged region from surrounding connective tissues so that periodontal tissues are smoothly regenerated. In other words, new periodontal tissues are regenerated by isolating the damaged region from external environment with the membrane so that invasion of gum fibroblasts are prevented and cells of a bone and periodontal ligament having regenerating ability in the tissues are regenerated without interference.

In the beginning of research on membranes, non-degradable materials such as polytetrafluoroethylene, cellulose acetate, silicon rubber, and polyurethane were used. However, a membrane made with the non-degradable materials has problems such as requirement of a secondary operation to remove the membrane after regeneration of a periodontal bone, unnecessary inflammation or tissue necrosis occurring in the treatment, and occurrence of abscess in new tissue, epithelial down growth, formation of paradental cyst and inflammation.

Recently, researches utilizing aliphatic polyester or biodegradable polymer such as collagen have been reported. It has been reported that re-operation is not required to remove a membrane, if a biodegradable membrane is used, and does not show any big difference in tissue regeneration compared to a membrane made of a non-degradable material. However, in the case that a membrane manufactured with the biodegradable material is applied to clinical treatment, there is a problem of secondary inflammation, because it can not maintain a fixed shape due to its low strength, and can not secure enough space for tissue growth.

Accordingly, a membrane should have a strength and structure to maintain a space for tissue growth of a periodontal bone. The membrane should also have biocompatibility with bone cells to induce fixation and growth of the bone cells when applied to a region of a damaged periodontal bone, and have porosity to effectively transport nutrients and water.

As a part of such researches, it has been reported that a membrane is prepared by applying drugs and biodegradable polymers selected from the group consisting of lactic acid homopolymer, copolymer of lactic acid and glycolic acid, or a mixture thereof, to a gauze made of polyglycolic acid (Korea Patent No.0180585). The membrane is manufactured by the steps of: applying a polymer solution containing a biodegradable polymer to a polyglycolic acid gauze, extracting the polymer by evaporating a solvent, forming micropores in the spaces of the gauze by fixing and stretching the extracted polymer on the polyglycolic acid gauze. However, the size of the micropores formed in the membrane should be controlled, because periodontal tissues and connective tissues should be isolated so that regeneration of periodontal tissues is effectively induced during the application of the membrane.

Additionally, it has been disclosed that a membrane for guided tissue regeneration is manufactured by utilizing chitosan of a natural polymer and biodegradable polymer of a synthetic polymer (Korea Patent Publication No. 2003-2224). This membrane is manufactured by forming a polymer film through applying a biodegradable polymer solution to a nonwoven made of the chitosan, and laminating another nonwoven made of the chitosan thereto. In the membrane, the nonwoven made with the biodegradable polymer has micropores to provide a condition for periodontal bone growth, and mechanical strength may be improved by laminating the nonwovens repeatedly. However, the membrane has a disadvantage that the manufacturing process is complicated, because the membrane is manufactured by the steps of: preparing a nonwoven with chitosan, forming a polymer membrane by applying a biodegradable polymer solution to the nonwoven, and laminating the nonwovens made of the chitosan.

Natural silk fiber is a fiber obtained from silkworms. Silk has been used as a high quality fiber material, because it has characteristics such as high tensile strength, peculiar luster, and excellent dyability. A silk fiber has a structure that two strands of fibroin are surrounded by a sericin wall. By preparing in various forms such as a membrane, powder, gel, and aqueous solution, the silk fibroin is used in various fields such as foods, cosmetics, and medical goods, because it has excellent biocompatibility and doesn't give any adverse effect to surrounding tissues.

Additionally, the silk fibroin is biocompatible and its powder is useful as a substance for growing or activating epidermal cells. Further, micropowder of the silk fibroin is used as a filler, coating agent, or cosmetic substance. Powder used for cosmetics or paints is prepared by removing sericin from natural silk fibers, reducing molecular weight by alkali, and by grinding. A method of preparing the powder is disclosed in Korea Patent Publication No. 2001-52075, and it has been reported that silk fibroin powder having a diameter less than 3 micrometers gives excellent moisture absorption, moisture-proof property, and moisture permeability.

Additionally, U.S. Pat. No. 6,110,590 disclosed a method of obtaining a silk nanofiber nonwoven by dissolving silk fibers in hexafluoroisopropanol without any pretreatment, and by electrospinning. However, this method has disadvantages that biocompatibility is reduced because sericin is not removed from the silk fibers and, particularly, it is difficult to commercialize because it takes several months to dissolve the silk.

### SUMMARY OF THE INVENTION

An object of the present invention is to solve the aforementioned problems, and to provide a nanofibrous nonwoven membrane containing silk fibroin for guided bone tissue regeneration, which has a predetermined strength, biocompatibility, and biodegradability, and is manufactured by a simple process with easy control of micropore size, and the manufacturing method thereof.

In order to achieve the above object, the present invention provides a nanofibrous nonwoven membrane containing silk fibroin for guided bone tissue regeneration, having a structure that nanofibers of silk fibroin obtained by removing sericin from silk fibers are formed as a nonwoven.

Additionally, in order to achieve the above object, the present invention provides a manufacturing method of a nanofibrous nonwoven membrane containing silk fibroin for guided bone tissue regeneration, including the steps of: rapidly freezing a silk fibroin solution obtained by removing sericin from silk fibers, drying, dissolving the dried silk fibroin in an electrospinning solvent, and by electrospinning.

### BRIEF DESCRIPTION OF THE DRAWINGS

FIG. 1 is a schematic view showing a manufacturing device of a membrane for guided bone tissue regeneration according to the present invention.

FIG. 2 is a micrograph of scanning electron microscopy showing a surface of a membrane for guided bone tissue regeneration according to Example 2 of the present invention.

FIG. 3 is a graph showing a distribution of diameters of silk fibroin ultra-micro fibers according to the present invention.

FIG. 4 is micrographs of scanning electron microscopy showing aspects of osteoblast fixed to a membrane for guided bone tissue regeneration.

FIG. 5 is a photo of a tissue sample observed with a low magnifying power (20×), taken 4 weeks after grafting a membrane for guided bone tissue regeneration onto a damaged region of rabbit skull.

FIG. 6 is a photo of a tissue sample observed with a high magnifying power (100×), taken 4 weeks after grafting a membrane for guided bone tissue regeneration onto a damaged region of rabbit skull.

### DETAILED DESCRIPTION OF THE INVENTION

In the description of the present invention, “nanofiber” indicates a fiber having a diameter of nanometers, and a nanofiber having a diameter of 100-1,000 nm may easily be manufactured by controlling the condition of electrospinning. Additionally, in the description of the present invention, “electrospinning solvent” indicates a solvent applicable to electrospinning, which can dissolve silk fibroin.

Silk fibroin constituting a membrane for guided bone tissue regeneration according to the present invention meets the requirements for the membrane such as affinity to biological tissues, biodegradability, permeability, impregnation of drugs such as antibiotics, and convenience in use. Additionally, the silk fibroin maintains mechanical characteristics during the manufacturing of nanofibers. Therefore, stability of the nanofibers is increased, porosity and shape of a nonwoven may be uniformly maintained during the manufacturing process, and the nonwoven can sufficiently sustain the pressure applied to a damaged region.

An electrospinning solvent to dissolve freeze-dried silk fibroin is preferably selected from the group consisting of: 1,1,1,3,3,3-hexafluoroisopropanol, a hydrate of 1,1,1,3,3,3-hexafluoroisopropanol, 1,1,1,3,3,3-hexafluoroacetone, a hydrate of 1,1,1,3,3,3-hexafluoroacetone, formic acid, or a mixture thereof. However, the electrospinning solvent is not limited to the above examples. Silk fibroin is preferably added in an amount of 5-15% by weight of 1,1,1,3,3,3-hexafluoroisopropanol, a hydrate of 1,1,1,3,3,3-hexafluoroisopropanol, 1,1,1,3,3,3-hexafluoroacetone, a hydrate of 1,1,1,3,3,3-hexafluoroacetone, or preferably 5-20% by weight of the formic acid.

A manufacturing method of a membrane for guided bone tissue regeneration includes the steps of: dialyzing, rapidly freezing, and drying a silk fibroin solution obtained by removing sericin from silk fibers; dissolving the dried silk fibroin in an electrospinning solvent; and electrospinning. The manufacturing method according to the present invention may further reduce water solubility and increase mechanical strength by performing recrystallization of silk fibroin nanofibers. C₁˜C₃ alcohol such as methanol, ethanol, propanol, or isopropanol or its aqueous solution may be used as a solvent for the recrystallization.

Natural silk fiber obtained from silkworms has a structure that two strands of fibroin are surrounded by a sericin wall, and a process of removing sericin from silk fibers is called a scouring process. Methods for the scouring process are a technology known to those skilled in the art. For example, there are many scouring methods such as a scouring utilizing protein decomposing enzyme such as Asperdillus oryzae, a scouring by boiling in alkali solution such as sodium carbonate and sodium oleate, and high temperature-high pressure scouring utilizing an autoclave. If a subsequent process is carried out without removing sericin, a large amount of foam is generated, and many problems may thereby occur in the subsequent process.

A silk fibroin solution is prepared by dissolving the sericin-removed silk fibroin in a proper solvent. A method of obtaining the silk fibroin solution is also a technology known in the art. For example, the solution is prepared by the steps of: dissolving silk fibroin in an ethanol solution containing neutral salts such as lithium chloride, lithium bromide, sodium iodide, zinc chloride or calcium chloride; dialyzing the solution by utilizing a dialysis membrane such as a cellophane; and completely removing the neutral salts.

Nanofibers constituting the membrane for guided bone tissue regeneration are manufactured by dissolving freeze-dried silk fibroin formed as a sponge after the dialyzing process in an electrospinning solvent, supplying the solution to an electrospinning device, and performing electrospinning.

An electrospinning device applicable to the electrospinning process is particularly not limited, and may properly be selected by considering the diameter and thickness of a nanofiber. An electrospinning device capable of applying a high voltage (5˜50 kV) may generally be used.

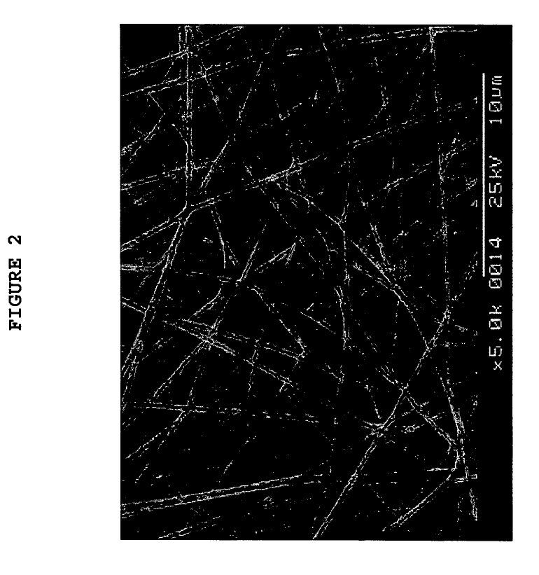

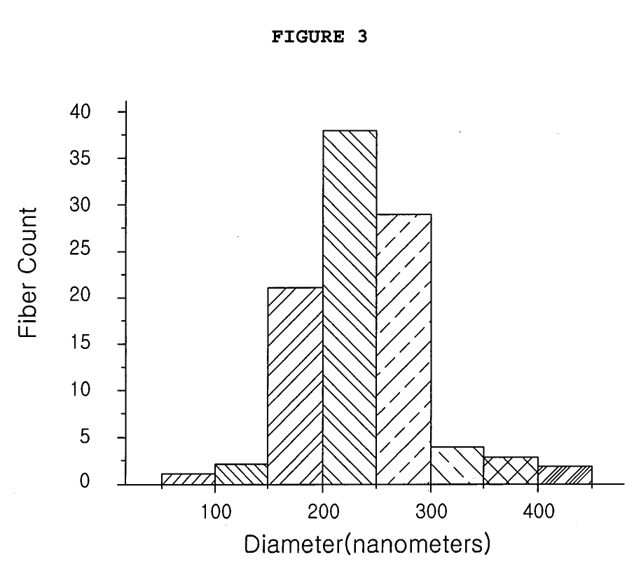

According to a concentration of silk fibroin solution, type of an electrospinning device, and electrospinning condition, the diameter of a nanofiber is controlled in the range of 100-1,000 nm, preferably in the range of 100-500 nm, and most preferably in the range of 100-300 nm. This technology is known to those skilled in the art. According to an exemplary embodiment of the present invention, addition of silk fibroin is preferably 5˜20% by weight of formic acid solution, and more preferably 8˜10%. Additionally, voltages is preferably applied in the range of 5˜35 kV, and more preferably in the range of 15˜25 kV. The distance between a spinneret and collector screen is preferably 5˜30 cm, and more preferably 5˜15 cm. Concentration of the silk fibroin solution, voltage, and the distance between the spinneret and the collector screen have to be determined by totally considering the type of an electrospinning device, required fiber properties, and structure of a membrane. Electrospun nanofibers form a nonwoven having fibers entangled (FIGS. 2 and 3).

A membrane for guided bone tissue regeneration according to the present invention may be modified according to the condition of use. A thickness of a membrane may be adjusted by controlling the fineness of the nanofibers and accumulation of nanofibers, and pore sizes may also be adjusted in a nonwoven manufacturing process. The thickness of the membrane is preferably 0.1˜5 mm and a pore size is preferably 2˜10 μm. However, the present invention is not limited thereto.

In more detail, fineness of the nanofibers may be adjusted by controlling a spinneret diameter of an electrospinning device extruding a silk fibroin solution, spinning speed, voltage, electric field, property of the polymer, and concentration of a polymer. The fineness of the nanofiber is preferably 0.001˜10 μm. However, the present invention is not limited thereto.

Additionally, porosity and compactness of the nanofibers may be controlled by adjusting accumulation time, voltage, and distance between the spinneret and collector screen.

Additionally, a membrane for guided bone tissue regeneration according to the present invention is manufactured in a nonwoven form from silk fibroin having biodegradability and biocompatibility and, particularly, the nanofibous nonwoven may be manufactured without any additional treatment. Accordingly, the membrane may be simply manufactured without application of biopolymer after forming a basic structure of the membrane.

A membrane for guided bone tissue regeneration according to the present invention may further include an additive used conventionally for a membrane, such as a drug, growth factor, ceramic, and enzyme.

A drug may include antibiotics for reducing inflammation or drugs for curing periodontal diseases. A drug for curing periodontal diseases is selected from the group consisting of: mefenamic acid, ibuprofen, flubiprofen, indomethacin, naproxen, metronidazole, tetracycline, minocycline, oxytetracycline, and a mixture thereof. The drugs for curing periodontal diseases may be contained in the membrane by dispersing in a polymer solution or preparing as an emulsion type, and by supplying to the electrospinning device. Alternatively, after manufacturing of a membrane, the membrane may be impregnated into a solution containing the drugs. However, the present invention is not limited to the above examples.

By adding the drugs to a membrane, the membrane for guided bone tissue regeneration according to the present invention maintains a sustained drug release system.

A growth factor is selected from the group consisting of: platelet-derived growth factor, insulin-like growth factor, epithelial growth factor, neoplatic growth factor, or a mixture thereof. The growth factor is added in the amount of 5˜20% by weight of silk fibroin polymer.

Hydroxyapatite, or tricalcium phosphate may be used as a ceramic. The ceramic is added to improve in vitro substrate component increasing biocompatibility and/or mechanical strength, and bone tissue regeneration effect. Particularly, since the hydroxyapatite has chemically and crystallographically similar characteristics as those of inorganic components in a bone or tooth, it has advantages that stability and fixation to surrounding bones or tissues are excellent when grafted into a human body. Accordingly, the membrane prepared by adding the hydroxyapatite is stabilized because it slowly releases the hydroxyapatite as growth of a bone proceeds.

Hereinafter, exemplary embodiments of the present invention will now be described in more detail. However, it should be understood that the invention is not limited to the embodiments herein disclosed. Various changes, substations and modifications may be made thereto by those skilled in the art without departing from the spirit or scope or the invention as described and defined by the appended claims.

### EXAMPLE 1

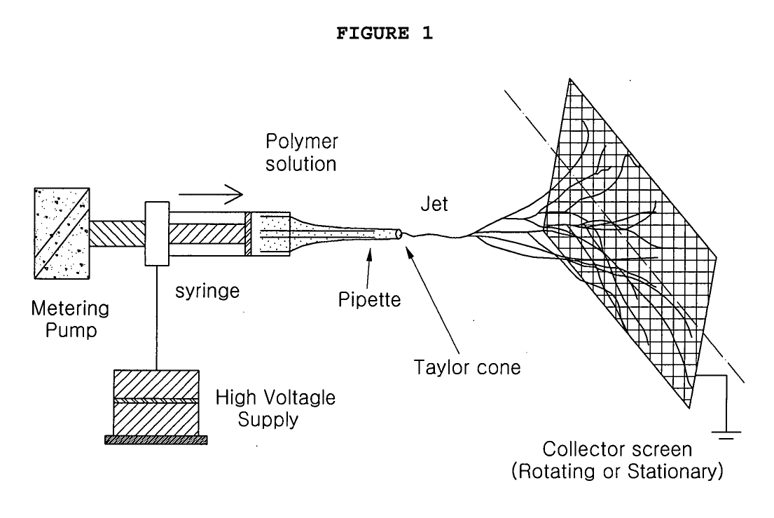

Silk fibers pre-washed with hot water were impregnated in water having the weight of 100 times of the fiber weight, and 0.3% sodium oleate by weight of the silk fiber was added to the silk fiber of the above quantity. After heating at 95° C. for 120 minutes and washing, the silk fiber was treated with 0.1% sodium oleate by weight of the silk fiber at 95° C. for 60 minutes. The solution was then neutralized with sodium carbonate solution and washed off several times with boiling water to completely remove sericin. The sericin-removed silk fibroin was added into a mixed solvent having mole ratio of calcium chloride:ethanol anhydrate:distilled water=1:2:8, and dissolved by agitating at 70° C. for 4 hours. The sericin-removed silk fibroin solution was then dialyzed with cellulose dialysis membrane for 3 days in the environment of distilled water to completely remove the salts and ethanol, and a pure silk fibroin solution was obtained. Dry silk fibroin in a sponge form was obtained by rapidly freezing the silk fibroin solution at −80° C. after removal of the salts and ethanol, and drying at −4° C. in a freeze dryer for 2 days. A 9% silk fibroin solution was prepared by dissolving the dry silk fibroin in formic acid. An aggregate of silk fibroin nanofibers was obtained by electrospinning with an electrospinning device shown in FIG. 1, in the condition that the distance between a spinneret and collector screen is 5 cm and voltage is 15 kV. The manufactured nonwoven of ultra-micro fiber aggregate was crystallized by impregnating in methanol for 10 minutes. After completing the crystallization, a water-insoluble fiber aggregate was obtained by removing the methanol and water. An image analyzer (Scope Eye, Korea) was used to analyze diameters of the ultra-micro fibers constituting the fiber aggregate. FIG. 2 shows the result of observation of the fiber aggregate with a scanning electron microscope (Hitachi S-2350, Japan) having 5,000 magnifying power. FIG. 3 shows that the distribution of fibers is concentrated in the range of 150˜300 nm. As described above, the fiber aggregate has nanofibers having a relatively uniform fineness of 150˜300 nm and a nonwoven structure of entangled fibers.

### EXAMPLE 2

Procedures of this example was carried out with the same method as Example 1, except that the electrospinning was performed by setting the distance between the spinneret and collector screen at 7 cm and voltage at 20 kV. An aggregate of ultra-micro fiber having relatively uniform fineness (210±140 nm) was obtained.

### EXAMPLE 3

Procedures for this example was carried out with the same method as Example 1, except that silk fibroin is dissolved in 1,1,1,3,3,3-hexafluoroisoporpanol to obtain a uniform 7% solution, and the electrospinning was performed by setting the distance between the spinneret and collector screen at 7 cm. An aggregate of ultra-micro fiber having relatively uniform fineness (230±150 nm) was obtained.

Experiment 1

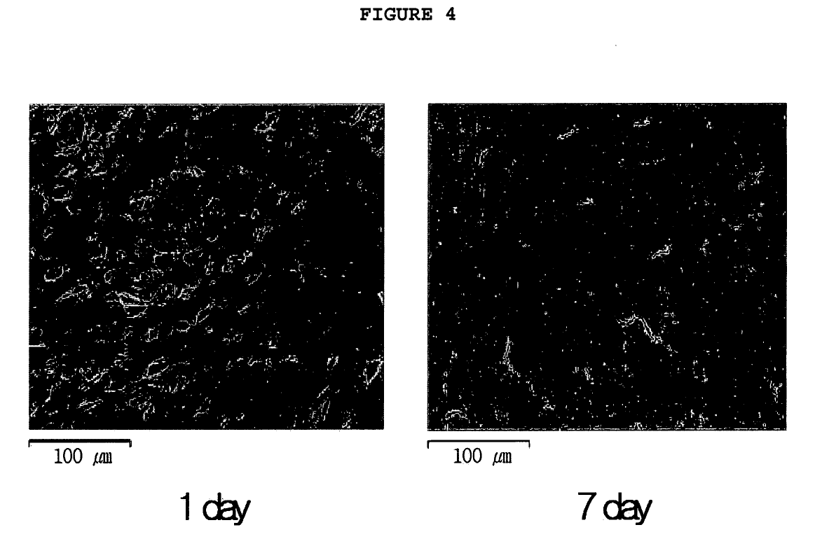

Cultured osteoblast was attached to a circular membrane formed with an ultra-micro fiber aggregate and having a diameter of 8 mm, and the extent and shape of fixation were observed with a scanning electron microscope after 1 day and 7 days. After 1 day, cells were evenly attached to the membrane maintaining its natural pyramidal form. After 7 days, most of the membrane was covered with the osteoblast (FIG. 4).

Experiment 2

A membrane made of the ultra-micro fiber aggregate was transplanted on the upper region of a drilled rabbit skull. The rabbit was sacrificed after 4 weeks, and bone bridge formation beneath the membrane was observed.

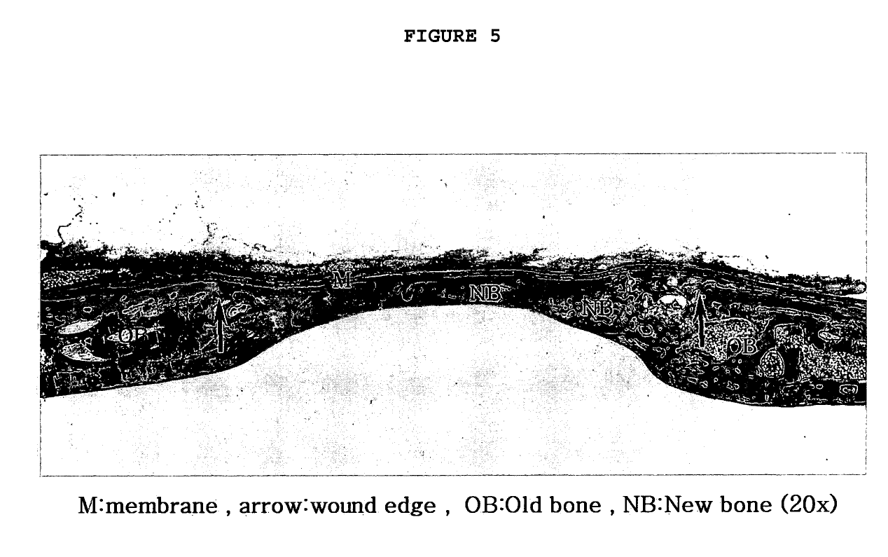

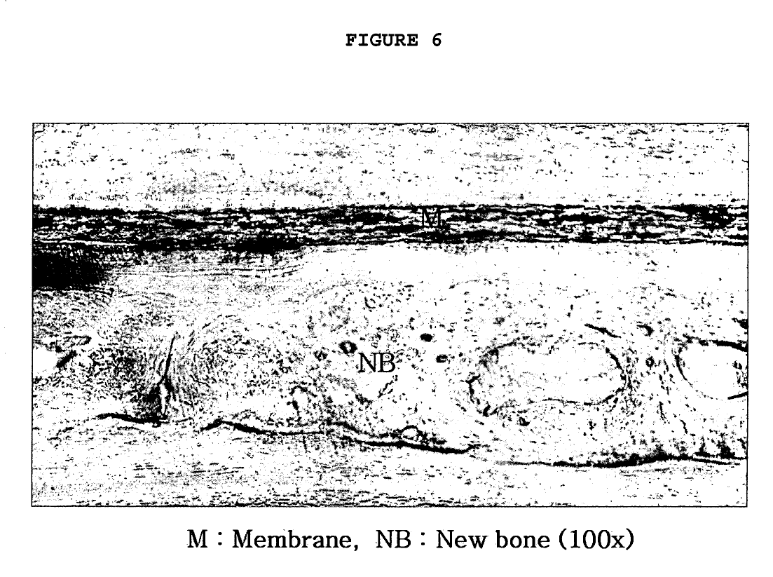

According to histological observation after 4 weeks, new bones and bone bridges were formed in the whole damaged bone area underneath the membrane, the new bones were growing up at the edges of the damaged region, and bone fusion with the original skull was nicely attained (FIG. 5). In a high power microscopic observation, it has been identified that thick bone is formed in the periphery of the damaged bone region beneath the membrane, showing a significant new bone formation developed from osteoid form. Bone formation in the center of the damaged region showed a round osteoid form connecting each other (FIG. 6). Additionally, although slight decomposition of the membrane was observed 4 weeks after transplantation, the membrane maintained nearly its initial shape of the transplantation.

As described above, a membrane for guided bone tissue regeneration according to the present invention has a predetermined strength, biocompatibility, and biodegradability, and may maintain a sustained drug release system, when drugs are added in the manufacturing process. Additionally, the membrane for guided bone tissue regeneration according to the present invention may be modified corresponding to the condition of usage, because a thickness of the membrane may be adjusted by controlling the fineness and compactness of nanofibers, and pore size of a multiporous structure may be adjusted, in a nonwoven manufacturing process. Additionally, the nanofibrous membrane for guided bone tissue regeneration according to the present invention may simply be manufactured from silk fibroin in a single step, without a laminating process.

The entire disclosure of Korean Patent Application No. 10-2004-0087254 filed on Oct. 29, 2004 including specification, claims, drawings and summary are incorporated herein by reference in its entirety.

What is claimed is:

1. A membrane for guided bone tissue regeneration having a porous structure of a nonwoven made of silk fibroin nanofibers obtained by removing sericin from silk fibers.

2. The membrane for guided bone tissue regeneration of claim 1, wherein the membrane further includes an additive.

3. The membrane for guided bone tissue regeneration of claim 2, wherein the additive is selected from the group consisting of a drug, a growth factor, a ceramic, and a mixture thereof.

4. The membrane for guided bone tissue regeneration of claim 1, wherein the pore size of the porous structure is 2˜10 μm.

5. The membrane for guided bone tissue regeneration of claim 1, wherein the thickness of the membrane is 0.1˜5 mm.

6. The membrane for guided bone tissue regeneration of claim 1, wherein the thickness of the nanofiber is 0.001˜10 μm.

7. A manufacturing method of a membrane for guided bone tissue regeneration, the membrane having a porous structure of a nonwoven made of silk fibroin nanofibers obtained by removing sericin from silk fibers, the method including the steps of:
dialyzing, rapidly freezing, and drying a silk fibroin solution obtained by removing sericin from silk fibers; and
electrospinning after dissolving the dried silk fibroin in an electrospinning solvent.

8. The manufacturing method of claim 7, wherein the solvent is selected from the group consisting of:
1,1,1,3,3,3-hexafluoroisopropanol, a hydrate of 1,1,1,3,3,3-hexafluoroisopropanol, 1,1,1,3,3,3-hexafluoroacetone, a hydrate of 1,1,1,3,3,3-hexafluoroacetone, formic acid, or a mixture thereof.

9. The manufacturing method of claim 7 further including a step of recrystallizing the silk fibroin nanofibers after the electrospinning.

10. The manufacturing method of claim 9, wherein the recrystallization is performed in C₁˜C₃ alcohol or aqueous solution thereof.
